(19) United States
(12) Patent Application Publication
(10) Pub. No.: US 2010/0310553 A1
(43) Pub. Date: Dec. 9, 2010

(54) Compositions and Methods for Controlling Hepatitis C Virus Infection

(76) Inventor: Guangxiang Luo, Lexington, KY (US)

Correspondence Address: Ashok Tankha, 36 Greenleigh Drive, Sewell, NJ, 08080, US

(21) Appl. No.: 12/438,137

(22) PCT Filed: Aug. 14, 2007
(86) PCT No.: PCT/US07/17970

§ 371 Date: Feb. 19, 2009

Related U.S. Application Data
(60) Provisional application No. 60/822,354, filed on Aug. 14, 2006.

(51) Int. Cl. A61K 39/395; C07K 16/08; C07H 21/02; A61K 31/711; A61K 31/47; A61K 31/352; A61K 38/45; A61K 38/16; A61K 31/195; A61K 31/4709; G01N 33/53; A61P 31/14

(52) U.S. Cl. 424/133.1; 530/387.3; 530/388.3; 530/389.4; 424/149.1; 424/161.1; 536/24.5; 514/44 A; 514/313; 514/456; 424/94.5; 514/1.1; 514/562; 514/314; 435/7.2

(57) ABSTRACT

Disclosed herein are methods and compositions for the treatment and prevention of Hepatitis C Virus (HCV) infection and methods of screening for antiviral agents against HCV infection and/or production. A method of using compositions of certain apolipoprotein-specific monoclonal or polyclonal antibodies to inhibit HCV infectivity is disclosed. Further, methods of using small interfering RNAs (siRNAs) specific to apolipoproteins for treating and/or preventing HCV infection are disclosed. Also disclosed are methods of using siRNAs specific and/or small molecule inhibitors to certain lipoprotein biosynthetic genes and of using recombinant apolipoprotein E and/or their forms of lipoproteins to treat and/or prevent HCV infections. Screening methods for anti-HCV agents include assessing the effect of a candidate agent on apolipoprotein E and/or apolipoprotein C-I gene expression, assembly, and/or secretion and assessing the effect of a candidate agent on the blockage of the interaction and/or incorporation of HCV nonstructural proteins and/or their fusion forms with reporter proteins into HCV virions.

This application claims the benefit of provisional application No. 60/822,354 titled “Composition And Methods For Treating And Preventing Hepatitis C Virus Infection and Screening Methods For Identifying Anti-Hepatitis C Virus Agents” filed Aug. 14, 2006.

### STATEMENT REGARDING FEDERALLY SPONSORED RESEARCH

Research for this invention was made with support from the NCI, Grant Number CA093712, and the NIAID, Grant Number AI51592.

### FIELD OF TILE INVENTION

The present invention relates to the treatment and prevention of Hepatitis C Virus (HCV) infection and screening for antiviral agents against HCV infection and/or production.

### BACKGROUND OF THE INVENTION

The hepatitis C virus (HCV) was discovered in 1989 by molecular cloning and has since been recognized as a major cause of viral hepatitis in humans. HCV is a single-stranded positive-sense RNA virus, which is about 9.6 kb in length. HCV belongs to the Hepacivirus genus of the family Flaviviridae. The viral RNA genome consists of the 5′ untranslated region (5′UTR), a single open reading frame (ORF) encoding a viral polypeptide of 3,010-3,040 amino acids, and the 3′ untranslated region (3′UTR) of variable length. Upon translation, the viral polyprotein is cleaved by cellular peptidases and viral proteases into core (C), envelope glycoproteins (E1 and E2), P7, non-structural (NS) proteins NS2, NS3, NS4A, NS4B, NS5A, and NSSB. Sequence analysis and comparison studies have revealed that both the 5′UTR and 3′UTR of the HCV genome are highly conserved. In contrast, sequences of the ORF exhibit a variation among HCV isolates. Based on the nucleotide sequence similarity, HCV has been further grouped into six major genotypes and numerous subtypes.

HCV infection is characterized by the establishment of chronic infection in up to about 85% of individuals exposed to HCV. The chronic HCV infection carries an increased risk of developing fatal liver diseases such as cirrhosis, liver failure, and hepatocellular carcinoma. HCV-associated end-stage liver disease is the leading cause of liver transplantation in the United States (US). It is estimated that approximately 4 million people in the US and 170 million people worldwide are persistently infected with HCV. Each year, HCV infection results in 8,000-10,000 deaths in the US alone. HCV-related deaths are expected to triple within the next 10-20 years if no effective intervention is made available. Currently, there is no specific and effective therapy to treat HCV infection. Accordingly, there remains an urgent need in the art for specific antiviral targets and agents for effectively treating and preventing HCV infection.

The structure and biochemical compositions of HCV virions have not been determined, although certain studies have found that HCV virions isolated from the plasma of hepatitis C patients were associated with lipoproteins to form lipoviroparticles (LVPs). Apolipoproteins B and E were detected in the low-density fractions of HCV RNA-containing particles, which could also be captured by apolipoprotein-specific antibodies, suggesting an association of the low-density HCV virions with human lipoproteins. However, the roles of apolipoproteins in HCV assembly and production have not been defined.

### SUMMARY OF THE INVENTION

The present invention addresses the above identified problems, and others, by providing compositions and methods for treating and/or preventing hepatitis C virus infection in humans. The present invention further includes targets and methods for identification (screening) of effective anti-HCV agents.

The present invention discloses a method of using compositions of apoE- and/or apoC-I-specific monoclonal or polyclonal antibodies to inhibit HCV infectivity. The method further comprises the step of administering an effective amount of the composition to a patient.

The present invention discloses methods of using siRNAs specific to apolipoproteins, for treating and/or preventing HCV infection. The present invention further includes siRNAs specific for certain lipoprotein biosynthetic genes for treating and/or preventing HCV infection. Also disclosed is a method of using recombinant apoE (E2, E3, and E4) protein and/or their forms of lipoproteins to treat and/or prevent HCV infections.

The present invention further discloses a method of screening for anti-HCV agents by assessing the effect of a candidate agent on apoE and/or apoC-I gene expression, assembly, and/or secretion. The present invention also includes a method of screening for anti-HCV agents by assessing the effect of a candidate agent on the blockage of the interaction and/or incorporation of HCV nonstructural proteins and/or their fusion forms with reporter proteins into HCV virions.

### BRIEF DESCRIPTION OF THE DRAWINGS

The foregoing summary, as well as the following detailed description of the embodiments, is better understood when read in conjunction with the appended drawings. For the purpose of illustrating the invention, exemplary illustrations of the invention are shown in the drawings. However, the invention is not limited to the specific methods disclosed herein.

FIG. 1 is a flow chart illustrating an exemplary method for preventing and/or treating Hepatitis C virus (HCV) infection in accordance with the present invention.

FIG. 2 is a flow chart illustrating an exemplary method of screening for anti-HCV agents in accordance with the present invention.

FIG. 3A depicts the results of HCV virion RNA (vRNA) determined by a ribonuclease (RNase) protection assay (RPA) in a sucrose gradient sedimentation analysis of HCV virions in culture media and densities (g/ml) of each fraction.

FIG. 3B is a Western blot analysis of HCV NS3 protein in cells infected with the different fractions identified in FIG. 3A.

FIG. 3C depicts the results of RPA used to determine positive-strand HCV RNA in cells infected with the different fractions identified in FIG. 3A.

FIG. 3D is a Western blot analysis of apolipoproteins B100 (apoB100), C-I (apoC-I), and E (apoE), wherein a density gradient sedimentation analysis of HCV RNA-containing particles is performed as shown in FIG. 3A, and apoB100, apoC-I, and apoE proteins were detected by using apoB-, apoCI- and apoE-specific antibodies.

FIG. 4A depicts the results of a study carried out to determine the HCV-neutralizing activity of apoE-specific monoclonal antibodies, where HCV positive-strand RNA levels in the Huh7.5 cells infected with HCV in the presence of apoE-specific monoclonal antibodies were determined by RPA.

FIG. 4B is a graph showing the correlation between HCV-neutralizing activity and concentrations of apoE-specific monoclonal antibodies using the quantitative data derived from FIG. 4A.

FIG. 4C shows the reduction of infectious HCV titers in the cell culture supernatant of Huh7.5 cells that were infected with HCV in the presence of apoE-specific monoclonal antibodies. The infectious HCV titers were determined by immunofluorescence assay (IFA) as foci-forming units per milliliter (ffu/ml) of cell culture supernatant.

FIG. 4D depicts the results of an analysis of the inhibition of HCV infectivity in the infectious fractions 3 to 5 of the sedimentation analysis as shown in FIGS. 3B and 3C by HCV E2- and ApoE-specific monoclonal antibodies.

FIG. 5 shows the reduction of HCV infectivity by an apoC-I polyclonal antibody, as determined by Western blot analysis of HCV NS3 protein in cells infected with HCV in the presence of increasing concentrations of apoC-I polyclonal antibody.

FIG. 6 indicates human apolipoprotein E gene sequences. The protein coding sequences are highlighted in bold letters and 50% gray in color, and the small interfering RNA (siRNA)-targeting sequences are shown in black color.

FIG. 7A depicts the results of a study conducted to determine the effects of ApoE-specific siRNA and a non-specific control (NSC) siRNA on suppression of HCV virion assembly and production, where the levels of ApoE and apoB proteins in the cell culture supernatants of the HCV-infected and siRNA-transfected cells were determined by Western blot analysis.

FIG. 7B depicts the correlation between siRNA concentrations and apoE levels in the cell culture supernatants using the quantified data derived from FIG. 7A.

FIG. 7C depicts the correlation between siRNA concentrations and apoB levels in the cell culture supernatants using the quantified data derived from FIG. 7A.

FIG. 8A depicts the results of a study conducted to determine the effects of ApoE-specific and NSC siRNAs on suppression of HCV virion assembly and production, where cell culture supernatant was used to infect naïve Huh7.5 cells; At 3 days post-infection, the levels of HCV NS3 protein and positive-strand RNA were determined by Western blot analysis and RPA, respectively.

FIG. 8B is a graph showing the correlation between siRNA concentrations and HCV positive-strand RNA levels relative to the control (without siRNA transfection) in Huh7.5 cells infected with the supernatants described in FIG. 8A. The data plotted in the graph are derived from the quantification of HCV positive-strand RNA levels in FIG. 8A.

FIG. 8C depicts the results of infectious HCV titers (ffu/ml) in the supernatants of the HCV-infected and apoE and NSC siRNA-transfected Huh7.5 cells.

FIG. 9A depicts the results of a study conducted to determine the effects of ApoE-specific and NSC siRNAs on suppression of HCV production, where the virion RNA (vRNA) levels of HCV RNA-containing particles secreted into the culture supernatants of the HCV-infected and apoE and NSC siRNA-transfected cells were determined by RPA.

FIG. 9B depicts the results of a study conducted to determine the effects of ApoE-specific and NSC siRNAs on suppression of HCV production, where HCV vRNA levels (%) are plotted against NSC and apoE siRNA concentrations (nM). The vRNA levels relative to control (without siRNA) are calculated as percentage of control (%) using the quantified data derived from FIG. 9A.

FIG. 10A depicts the results of a study conducted to determine the effects of ApoE-specific and NSC siRNAs on suppression of HCV assembly and production, where intracellular HCV virions were used to infect naïve Huh7.5 cells. At 3 days post-infection, the levels of HCV positive-strand RNA in the infected cells were determined by RPA.

FIG. 10B depicts the correlation between siRNA concentrations and HCV positive-strand RNA levels relative to the control (without siRNA transfection) in Huh7.5 cells infected with intracellular HCV virions described in FIG. 10A. The data plotted in the graph are derived from the quantification of HCV positive-strand RNA levels in FIG. 10A.

FIG. 10C depicts the results of infectious HCV titers (ffu/ml) of the intracellular HCV virions isolated from the HCV-infected and NSC and apoE siRNA-transfected Huh7.5 cells.

FIG. 11 depicts the results of a study conducted to determine the effects of NSC and ApoE-specific siRNAs on HCV RNA replication, where subgenomic HCV RNA-harboring cells were transfected with NSC and apoE siRNAs; and, at 3 days post-transfection, HCV positive-strand RNA levels were determined by RPA.

FIG. 12 depicts the results of a study to determine the effects of Sterol O-acyltransferase (SOAT or ACAT) and microsomal triglyceride transfer protein (MTP)-specific siRNAs on HCV production, wherein the HCV-infected Huh7.5 cells were transfected with increasing concentrations of NSC, ACAT, and MTP siRNAs, respectively, and, the cell culture supernatants were used to infect naïve Huh7.5 cells. At 3 days Post-infection, HCV RNA was extracted from cells with Trizol reagent, and HCV positive-strand RNA levels were determined by RPA.

FIG. 13 depicts the structures of CP-346086, glybenclamide, hesperetin, quercetin, and naringenin.

FIG. 14A depicts the results of a study conducted to determine the effects of CP-346086, a microsomal triglyceride transfer protein (MTP) inhibitor, on suppression of HCV production, where apoB100 and apoE levels in the supernatants of CP-346086-treated cells were determined by Western blot analysis.

FIG. 14B depicts the results of a study conducted to determine the effects of CP-346086 on suppression of HCV production, where the supernatants of CP-346086-treated and HCV-infected cells were used to infect naïve Huh7.5 cells. At 3 days post-infection, the levels of HCV NS3 protein was determined by Western blot analysis.

FIG. 14C depicts the results of a study conducted to determine the effects of CP-346086 on suppression of HCV production, where apoB, apoE, and HCV NS3 protein levels derived from quantification of FIGS. 13A and 13B are used to plot against CP-346086 concentrations (μM).

FIG. 14D depicts the results of a study conducted to determine the effects of CP-346086 on suppression of HCV production, where the supernatants of CP-346086-treated and HCV-infected cells were used to infect naïve Huh7.5 cells. At 3 days post-infection, the levels of HCV positive-strand RNA were determined by RPA.

FIG. 14E depicts the results of a study conducted to determine the effects of CP-346086 on suppression of HCV production, where infectious HCV titers (ffu/ml) in the cell culture supernatants of the CP-346086-treated cells were determined by IFA and are used to plot against CP346086 concentrations.

FIG. 14F depicts the results of a study conducted to determine the effects of CP-346086 on HCV RNA replication, where subgenomic HCV RNA-harboring cells were treated with CP-346086, and HCV positive-strand RNA levels in the cell were then determined by RPA.

FIG. 15 depicts the results of an RPA used to determine the effects of glybenclamide, hesperetin, and quercetin on HCV production; where the HCV-infected Huh7.5 cells were treated with increasing concentrations of glybenclamide, hesperetin, and quercetin, respectively; and, the cell culture supernatants were used to infect naïve Huh7.5 cells. At 3 days post-infection, HCV RNA was extracted with Trizol reagent, and HCV positive-strand RNA levels were determined by RPA.

FIG. 16A depicts the results of a Western blot analysis of HCV NS3 protein in a study to determine the inhibition of HCV infection by a recombinant apoE3 protein.

FIG. 16B depicts the results of a study to determine the effect of a purified apoC-I protein on the inhibition of HCV infection, where the levels of HCV positive-strand RNA in Huh7.5 cells infected with HCV in the presence of apoC-I protein were determined by RPA.

FIG. 17A is a Western blot analysis used to assess the inhibition of HCV infection by human high-density lipoproteins (HDL), low-density lipoproteins (LDL), and very low-density lipoproteins (VLDL).

FIG. 17B depicts the results of an RPA used to assess the inhibition of HCV infection by human HDL, LDL, and VLDL.

FIG. 18 is a Western blot analysis of HCV Core (C), NS3, and NS5A proteins, wherein a density gradient sedimentation analysis of HCV RNA-containing particles is performed as in FIG. 3A; The C, NS3, and NS5A were detected by Western blot analysis using C-, NS3-, and NS5A-specific monoclonal antibodies.

FIG. 19A depicts the construction of infectious HCV expressing an NS5A-GFP fusion protein and fluorescence photographs showing green fluorescence protein (GFP) expression in Huh7.5 cells infected with NS5A-GFP-expressing HCVs. GFP is inserted into NS5A (466 amino acids) at different positions indicated by amino acid numbers (247, 363, and 429).

FIG. 19B is a Western blot analysis of HCV NS5A or NS5A-GFP fusion protein expression in Huh7.5 cells infected with wild type HCV or mutant HCVs expressing NS5A-GFP fusion protein using NS5A- and GFP-specific monoclonal antibodies.

FIG. 19C is a Western blot analysis of the NS5A-GFP fusion protein present in HCV RNA-containing particles of the sucrose-gradient fractions using a GFP-specific monoclonal antibody.

### DESCRIPTION OF EMBODIMENTS OF THE INVENTION

The present invention includes compositions and methods for treating and/or preventing hepatitis C virus infection in humans. The present invention further includes targets and methods for identification (screening) of effective anti-HCV agents.

In accordance with the present invention, HCV assembly and/or HCV infectivity may be controlled by suppressing the levels of certain apolipoproteins (e.g., apoE and apoC-I) in patients, thereby treating and/or preventing hepatitis C virus infection. In accordance with the present invention, HCV may also be treated by targeting certain lipid and lipoprotein biosynthesis genes and/or pathways.

The present invention further includes methods of using monoclonal and/or polyclonal antibodies specific to apolipoprotein E and/or apolipoprotein C-I to treat and/or prevent HCV infection.

The present invention also includes methods of using siRNAs specific to apolipoproteins, including but not limited to apoE, for treating and/or preventing HCV infection. The present invention further includes siRNAs specific for certain lipoprotein biosynthetic genes, including: acyl coenzyme A:cholesterol acyltransferase (ACAT; also known as sterol O-acyltransferase (SOAT)), and microsomal triglyceride transfer protein (MTP), for treating and/or preventing HCV infection. Such siRNAs may be obtained, for example, from Dharmacon Company (Lafayette, Colo.).

The present invention also includes a method of using small molecular inhibitors of lipoprotein biosynthesis to treat and/or prevent HCV infections, for example, inhibitors of ACAT and MTP may be used. Examples of ACAT and MTP inhibitors include, but are not limited to: CP-346086, glybenclamide, hesperetin, quercetin, and naringenin.

The present invention additionally includes a method of using recombinant apoE (E2, E3, and E4) protein and/or their forms of lipoproteins to treat and/or prevent HCV infections, including high-density lipoproteins (HDL), low-density lipoproteins (LDL), and very low-density lipoproteins (VLDL).

The present invention further includes a method of screening for anti-HCV agents by assessing the effect of a candidate agent on apoE and/or apoC-I gene expression, assembly, and/or secretion.

The present invention also includes a method of screening for anti-HCV agents by assessing the effect of a candidate agent on the blockage of the interaction and/or incorporation of HCV nonstructural proteins (e.g., NS5A) and/or their fusion forms with reporter proteins (e.g., Luciferase, alkaline phosphotase, GFP, etc.) into HCV virions.

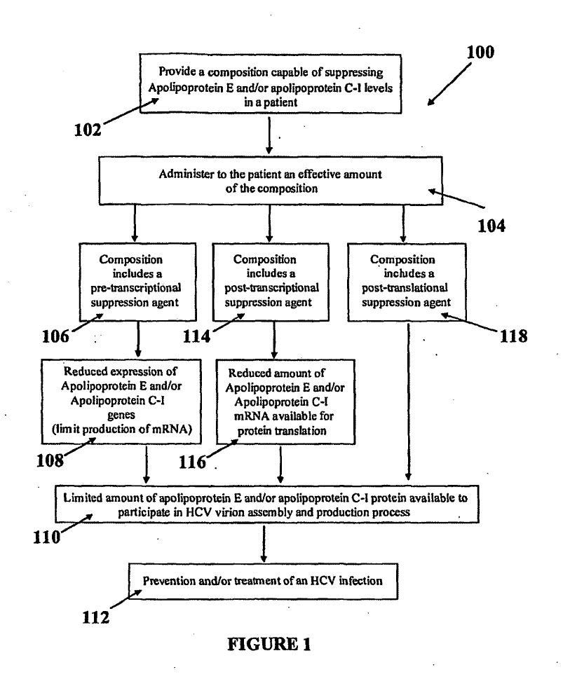

Turning now to FIG. 1, an exemplary method 100 of the present invention for treating and/or preventing HCV infection in a patient includes the following: providing a composition capable of suppressing apolipoprotein E (apoE) and/or apolipoprotein C-I (apoC-I) levels in the patient 102; and administering to the patient an effective amount of the composition 104. An effective amount of the composition to be used in accordance with the present invention is intended to mean a nontoxic but sufficient amount of the composition, such that the desired prophylactic or therapeutic effect is produced. The exact amount of the composition that is an effective amount will vary from patient to patient, depending, for example, on the age, and general condition of the patient, the severity of the infection being treated, the particular carrier or adjuvant being used and its mode of administration, and the like. Accordingly, the effective amount of any particular composition will vary based on the particular circumstances, and an appropriate effective amount may be determined in each case of application by one of ordinary skill in the art using only routine experimentation.

With continued reference to FIG. 1, the administered composition may include one or more agents capable of suppressing apoE and/or apoC-I. For example, apoE and/or apoC-I may be pre-transcriptionally suppressed 106, by providing a composition capable of decreasing apoE and/or apoC-I gene expression, such that the relative amount of apoE and/or apoC-I mRNA that is transcribed is decreased 108. By so reducing apoE and/or apoC-I gene expression, the amount of apoE and/or apoC-I protein that may be produced is decreased, thereby limiting the amount of apoE and/or apoC-I protein that is available to participate in the HCV assembly and production process 110, resulting in the prevention and/or treatment of an HCV infection in the patient 112. Examples of agents that may pre-transcriptionally suppress apoE and/or apoC-I include peroxisome proliferator-activated receptors (PPARs), antagonists and interferon.

ApoE and/or apoC-I may also be suppressed post-transcriptionally 114, for example, by providing a composition capable of degrading the apoE and/or apoC-I mRNA or otherwise blocking translation of apoE and/or apoC-I mRNA, thereby reducing the amount of apoE and/or apoC-I mRNA that is available for translation 116. By so reducing the amount of mRNA available for translation, the amount of apoE and/or apoC-I protein that may be produced is decreased, thereby limiting the amount of apoE and/or apoC-I protein that is available to participate in the HCV assembly and production process 110, resulting in the prevention and/or treatment of an HCV infection in the patient 112. Examples of agents that may post-transcriptionally suppress apoE and/or apoC-I include compounds capable of RNA interference, such as small interfering RNAs (siRNA). RNA interference is a post-transcriptional gene silencing tool, i.e., specifically degrading or destroying the mRNA encoding the product of the gene of interest. Short RNA duplexes that have been shown to successfully interfere with expression of specific genes of interest in cells are referred to as small interfering RNAs (siRNAs). An siRNA is a double stranded RNA oligonucleotide that is typically less than about 30 nucleotide bases in length. One strand of an siRNA is complementary to a portion of an mRNA of a gene of interest, binds thereto, and degrades mRNAs and/or prevents translation, thereby suppressing expression of the gene.

ApoE and/or apoC-I may additionally be suppressed post-translationally 118, for example, by providing a composition capable of degrading the apoE and/or apoC-I protein or scavenging the apoE and/or apoC-I protein, thereby limiting the amount of apoE and/or apoC-I protein that is available to participate in the HCV assembly and production process 110. By so limiting the amount of protein available for participation in the HCV assembly and production process, an HCV infection in the patient may be prevented and/or treated 112. Examples of agents that may post-translationally suppress apoE and/or apoC-I include acyl coenzyme A:cholesterol acyltransferase (ACAT) inhibitors (e.g., glybenclamide and naringenin), Cholesteryl Ester Transfer Protein (CETP) inhibitors (e.g., torcetrapib) and microsomal triglyceride transfer protein (MTP) inhibitors (e.g., CP346086 and BMS-200150). It is also possible that post-translational modification of apoE will affect its incorporation into HCV virions, for instance phosphorylation, and therein results in inhibition of HCV production.

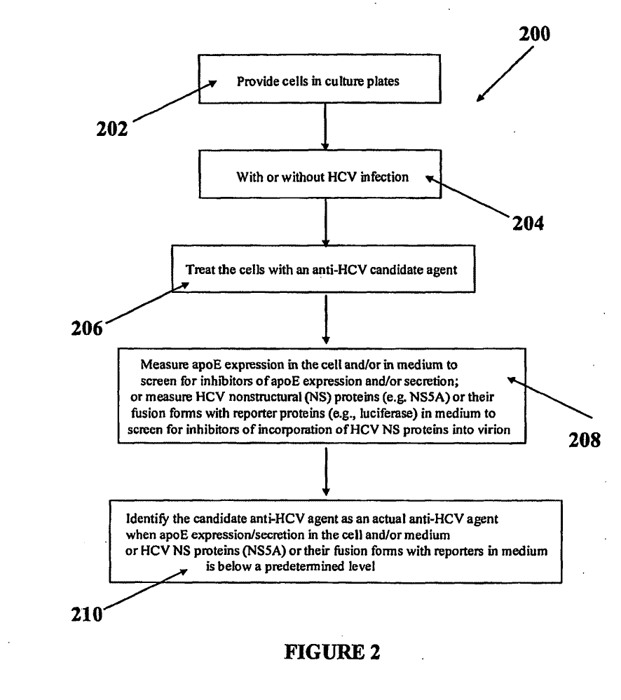

Turning now to FIG. 2, an exemplary screening method 200 of the present invention for identifying anti-HCV agents includes the following: providing cells in culture 202; uninfecting the cells, infecting the cells with HCV, or HCV-producing cells 204; treating the cells with a candidate anti-HCV agent 206; measuring apoE expression in the cells and/or cell culture supernatant or HCV nonstructural proteins (e.g., NS5A) and reporter gene (e.g., luciferase) that is fused with NS5A in the culture supernatant 208; and identifying the candidate agent as an actual anti-HCV agent when apoE gene expression in the cells and/or supernatant or HCV nonstructural proteins in the culture supernatant is below a predetermined level 210.

The cells that may be provided 202 include any cells that may be grown in culture, produce apolipoproteins, and/or are capable of being infected with HCV 204. The cells may be treated with a candidate anti-HCV agent 206 before and/or after infection with HCV, or without HCV infection, as desired. For example, if an assessment of the abilities of a candidate agent to prevent HCV infection is being made, it may be desirable to treat the cells with the candidate agent before infection with HCV. For another example, if an assessment of the abilities of a candidate agent to treat HCV infection is being made, it may be desirable to treat the cells with the candidate agent after infection with HCV. Additionally, cells without HCV infection can be treated with the candidate agent to assess its effect on apoE expression, assembly, and/or production (secretion).

The apoE and/or apoC-I gene expression level in the cells and/or in the culture supernatant or HCV nonstructural proteins and/or their fusion forms with reporter proteins (e.g., NS5A, NS5A-luciferase, NS5A-GFP, NS5A-alkaline phosphotase, and etc.) in the culture supernatant may be measured 208 by any method known to those skilled in the art. For example, the apoE and/or apoC-I gene expression level could be measured by quantifying the concentration of apoE and/or apoC-I mRNA. For another example, the apoE and/or apoC-I gene expression level could be measured by quantifying the concentration of apoE and/or apoC-I protein in the cells and/or in the cell culture supernatant. For another example, HCV nonstructural proteins (NS2, NS3, NS4A, NS4B, NS5A, and NS5B) in the cell culture supernatant of the HCV-infected cells could be measured by quantifying the levels of NS (e.g., NS5A) proteins and/or the levels of reporter activity (e.g., luciferase, alkaline phosphotase, GFP, and etc.) of the NS-reporter fusion proteins (e.g., NS5A-luciferase, NS5A-alkaline phosphotase, NS5A-GFP, and etc.) in the culture supernatant.

A candidate agent may be identified as an actual anti-HCV agent when the apoE and/or apoC-I gene expression level and/or HCV nonstructural protein (e.g., NS5A) and the reporter gene that is fused with HCV nonstructural protein (e.g., NS5A-luciferase) is below a predetermined level 210. The predetermined level may be identified, for example, by quantifying the apoE and/or apoC-I mRNA and/or protein in the cells and/or supernatant using methods known to those skilled in the art and comparing the quantified apoE and/or apoC-I mRNA and/or protein level to a standard curve plotting level of apoE and/or apoC-I mRNA and/or protein. For another example, the predetermined level may be identified using a control sample that does not contain the candidate anti-HCV agent. In this regard, when a control sample that does not contain the candidate anti-HCV agent is examined concurrently with a test sample that contains the candidate anti-HCV agent, the candidate anti-HCV agent is identified as an actual anti-HCV agent when the detected presence of apoE and/or apoC-I mRNA and/or protein in the test sample is less than the detected presence of apoE and/or apoC-I mRNA and/or protein in the control sample.

The present invention is further illustrated by the following specific but non-limiting examples. The following examples may include compilations of data that are representative of data gathered at various times during the course of developing and testing the efficacy of the present invention.

### EXAMPLES

In the studies described herein, the following is demonstrated: (1) HCV nonstructural proteins NS3 and NS5A or NS5A-reporter (e.g., green fluorescence protein, GFP) fusion protein are incorporated into infectious HCV particles; (2) apolipoproteins (B, C-I, and E) are present in HCV particles and the level of human apolipoprotein E correlate with that of HCV infectivity; (3) antibodies against apolipoprotein E and apolipoprotein C-I efficiently block HCV infection; (4) apoE-, ACAT-, and MTP-specific siRNAs each inhibit HCV production and/or infection; and (5) small molecular inhibitors of the lipid and lipoprotein biosynthesis pathways suppress HCV production.

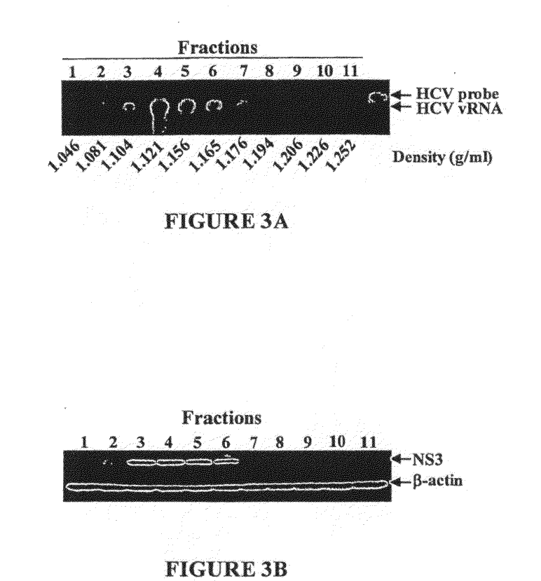

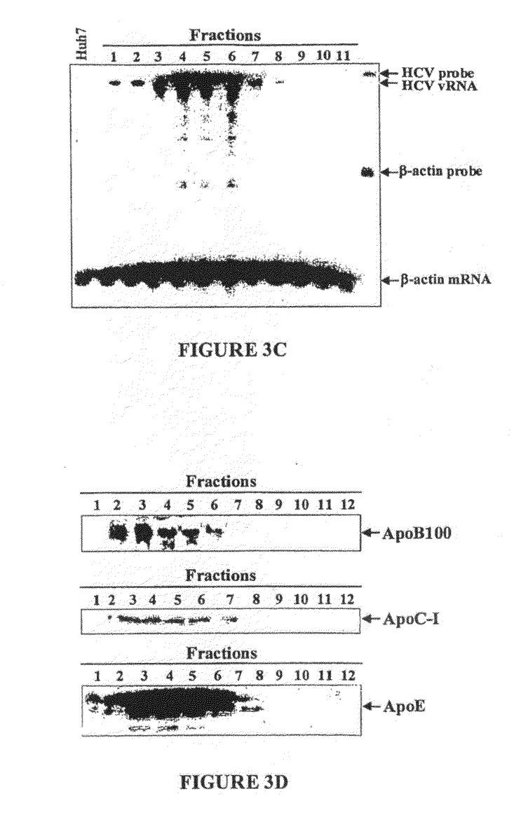

HCV virions contain human apolipoproteins (e.g., apoB, apoC-I, and apoE): To determine the properties of HCV RNA-containing particles, a continuous 20-60% sucrose density-gradient sedimentation analysis was performed. HCV RNA-containing particles in each fraction were measured by the level of HCV virion RNA (vRNA) and the HCV infectivity. With reference to FIG. 3A, which depicts the results of a sucrose gradient sedimentation analysis of HCV virions in the culture media, HCV virions were concentrated by ultracentrifugation and then fractionated through 20-60% continuous sucrose density-gradient centrifugation. Fractions (1 ml each) were collected from the bottom to the top. The HCV vRNA in each fraction, numbered (from the top to the bottom) on the top of FIG. 3A, was extracted with Trizol reagent and determined by an RNase Protection Assay (RPA). RPA is a laboratory test used to identify individual RNA molecules in a sample extracted from cells. The buoyant density (g/ml) of each fraction is indicated at the bottom of FIG. 3A. As shown in FIG. 3A, HCV vRNA was detected in nearly all fractions with peak levels in fractions 4 to 6. The buoyant density of the HCV RNA-containing particles covers a range varying from 1.05 to 1.25 g/ml. With reference to FIG. 3B, Western blot analysis of NS3 was conducted with cells infected by the various fractions. Huh7.5 cells in a multi-well culture plate were infected with 100 μl of each fraction. Cells were lysed at 3 days post infection, and the NS3 protein was detected by Western blot. With reference to FIG. 3C, RPA was used to determine positive-strand HCV RNA in cells infected with the different fractions. Results show that fractions 4, 5, and 6, which contain the highest levels of HCV vRNA, were of highest infectivity. These findings suggest that the lower-density particles on the top of the sucrose gradient were assembled in a form containing components necessary for HCV infectivity. The low density of HCV RNA-containing particles could be due to lipoproteins assembled with HCV virions.

To determine whether HCV RNA-containing particles produced by cell culture in vitro contain human apolipoproteins, Western blot analysis was performed using apoB-, apoC-I-, and apoE-specific antibodies. With reference to FIG. 3D, all apoB, apoC-I, and apoE were detected in the low-density fractions. More significantly, the levels of apoE in various fractions are similar to those of HCV vRNA and infectivity (reference to FIGS. 3A, 3B, and 3C). These findings suggest that HCV virions contain human apolipoproteins and that the levels of apoE correlate with the HCV infectivity.

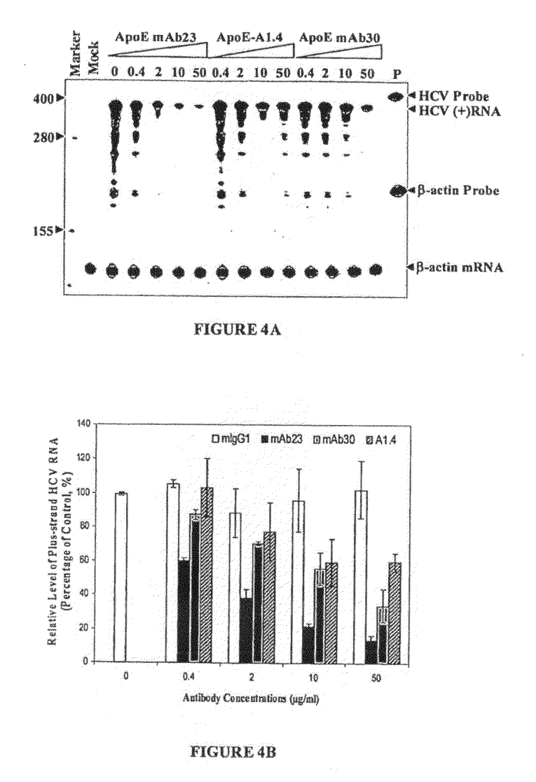

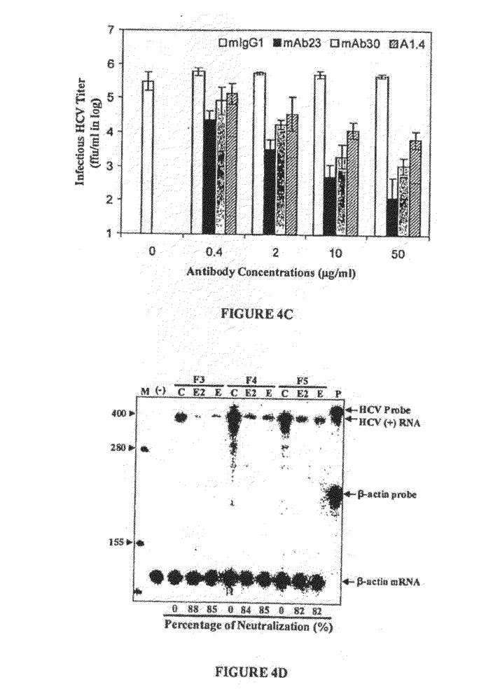

Inhibition of HCV infection by apolipoprotein E- and apoC-I-specific antibodies: As apolipoproteins are assembled in HCV virions, a study was conducted to determine whether apolipoprotein-specific monoclonal antibodies will block HCV infection. The inhibition of HCV infectivity by ApoE-specific monoclonal antibodies, apoC-I polyclonal antibody, and HCV E2-specific monoclonal antibodies was assessed. Huh7.5 cells were infected with HCV in the presence of increasing concentrations (μg/ml) of various apoE-specific monoclonal antibodies (mAb23, A1.4, and mAb30). At 3-hr post-infection (p.i.), HCV and apoE antibodies were removed, and the HCV-infected cells were washed with 1× phosphate buffered saline (1× PBS) twice. The HCV-infected Huh7.5 cells were then incubated with a cell culture medium, for example, Dulbecco's modified Eagle's medium (DMEM). After 3-day incubation, cell culture supernatants were collected and used for the determination of infectious HCV titer, while total cellular RNAs were extracted with Trizol reagent. With reference to FIG. 4A, the levels of HCV positive-strand RNA were determined by RPA using an α[³²P]UTP-labeled RNA probe containing a negative-strand HCV 3′UTR that is complimentary to positive-strand HCV RNA. FIGS. 4A and 4B show that apoE-specific monoclonal antibodies reduced HCV positive-strand RNA levels in a dose-dependent manner. ApoE monoclonal antibodies also decreased infectious HCV titers by nearly four orders of magnitude (10,000 times) at concentrations up to 50 μg/ml (mAb23 in black bar of FIG. 4C). With reference to FIG. 4D, HCV virions present in different fractions, i.e., fraction 3-fraction 5, as described in FIG. 3 above were incubated with antibodies specifically against apoE and HCV E2 during infection. Ten μg/ml of monoclonal antibodies against HCV E2 (CBH-5) and apoE were incubated with the fractions during infection. At 3-hr p.i., cells were washed twice with PBS and incubated with DMEM containing 10% fetal bovine serum. At 3 days p.i., total cellular RNAs were extracted with Trizol reagent. The levels of positive-stranded HCV RNA were determined by RPA. Results show that apoE-specific monoclonal antibody 23 (mAb23) potently blocked the infectivity of HCV virions in all different fractions, reducing HCV infectivity by more than 80% as highlighted by percentage of neutralization at the bottom of FIG. 4D. The sizes of RNA markers are indicated on the left and probes and protected RNA products are highlighted by arrows on the right. ApoE monoclonal antibody inhibited HCV infection at least as potently as HCV E2-specific monoclonal antibody (CBH5). Additionally, apoC-I-specific polyclonal antibody was assessed for its HCV-neutralizing capability. FIG. 5 shows that apoC-I antibody remarkably reduced the levels of HCV NS3 protein when present during infection, demonstrating its potent HCV-neutralizing activity. These findings suggest that antibodies against human apolipoproteins can be used as therapeutic and/or prophylactic agents to block HCV infection.

Suppression of HCV infectivity, assembly, and production by small interfering RNAs (siRNAs) against apolipoprotein E as well as lipoprotein biosynthetic genes: Acyl Coenzyme A:Cholesterol Acyltransferase (ACAT, also named Sterol O-acyltransferase-SOAT) and microsomal triglyceride transfer protein (MTP). To determine whether lipids and lipoproteins are required for HCV assembly and production, small interfering RNAs (siRNAs) were created, which siRNAs are included in certain exemplary compositions made in accordance with the present invention and can be used to practice certain exemplary methods in accordance with the present invention. The siRNAs described in this example are specifically against apoE, ACAT (SOAT), and MTP, which are required for lipoprotein synthesis, assembly and secretion.

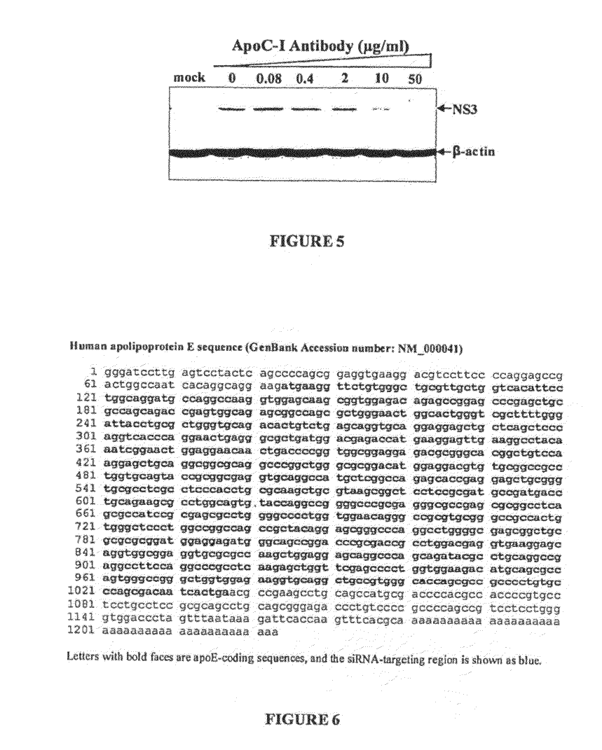

With references to FIG. 6, the human apolipoprotein E gene sequences (GenBank accession number: NM_{—}000041) are shown, with the protein-coding sequences in bold and 50% gray in color, and the siRNA targeting sequences in blue.

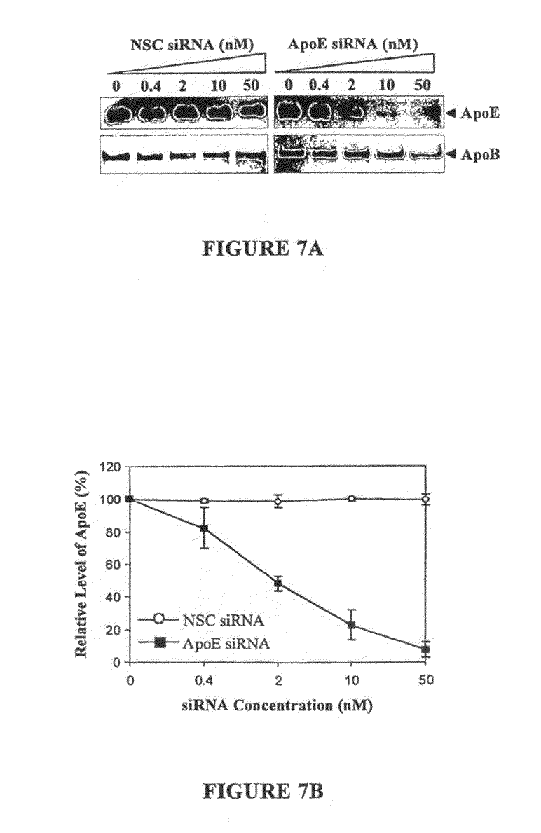

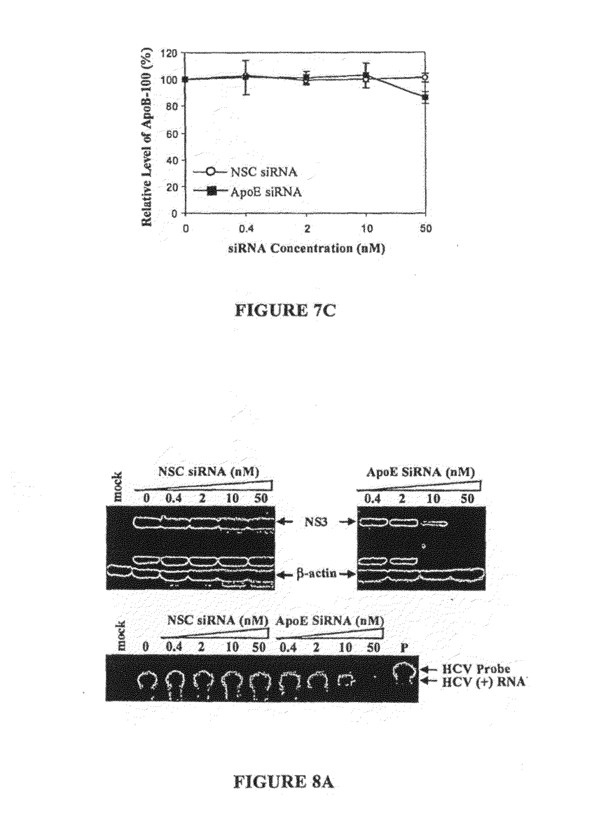

With reference to FIGS. 7A-7C, an apoE-specific siRNA and a non-specific control (NSC) siRNA were studied and apoB and apoE protein levels in the cell culture supernatants of siRNA-transfected cells were determined by Western blot analysis. Huh7.5 cells were infected with HCV and then transfected with siRNAs with apoE-specific or NSC siRNA at concentrations of 0.4, 2, 10, and 50 nM. The levels of apoB and apoE proteins secreted into the culture supernatants (media) were then quantified by Western blot analysis using apoB- and apoE-specific antibodies. FIG. 7 shows that ApoE-specific siRNA but not NSC siRNA efficiently reduced apoE expression by more than 90% at concentrations up to 50 nM in a dose-dependent manner. In contrast, apoB expression was unaffected by apoE-specific siRNA or NSC siRNA, demonstrating a specific inhibition of apoE expression by apoE-specific siRNA.

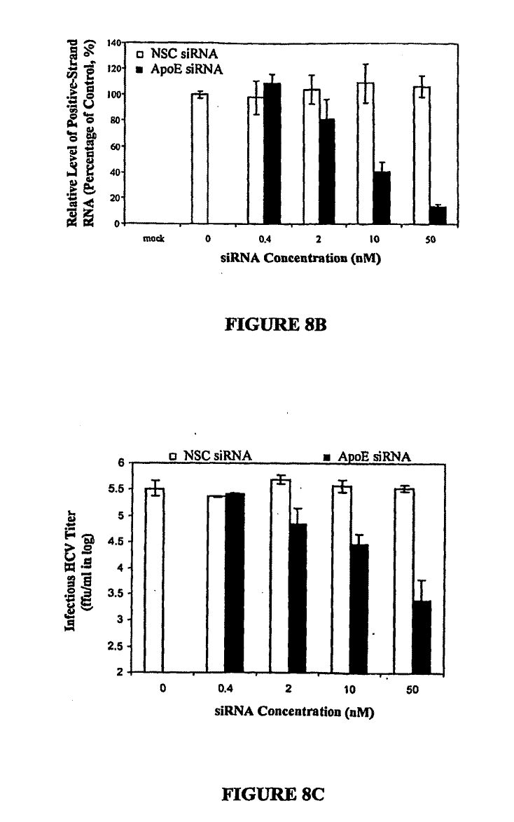

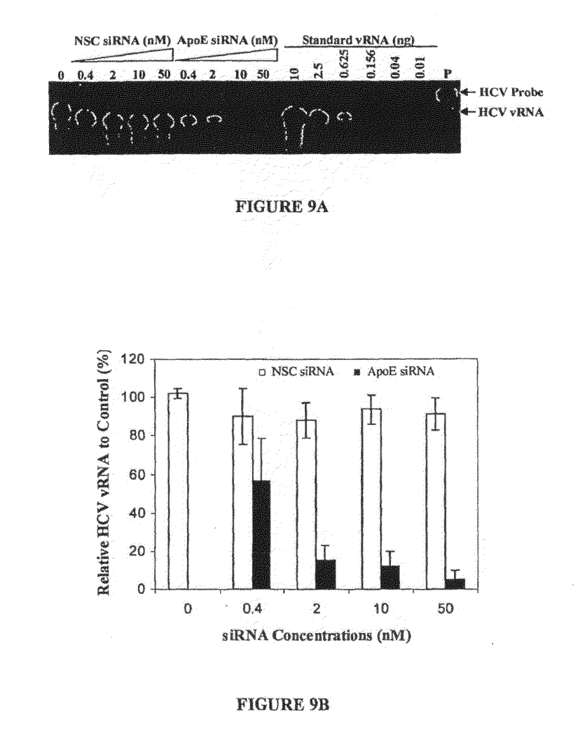

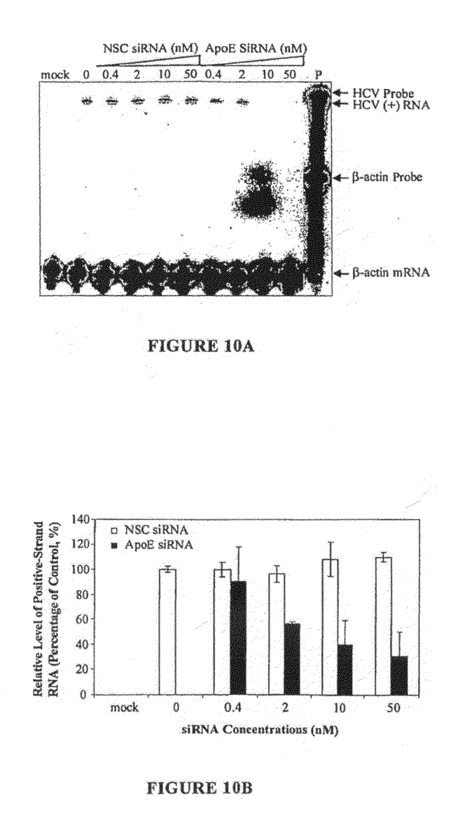

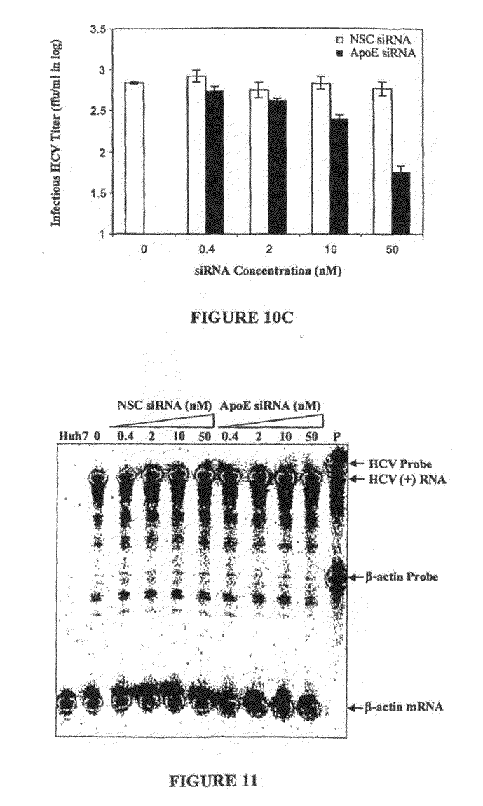

With reference to FIGS. 8A-8C, the effects of apoE siRNA on HCV production were studied by determining HCV infectivity of the cell culture supernatant derived from the HCV-infected and siRNA-transfected cells. Huh7.5 cells were infected with HCV and then transfected with apoE-specific siRNA or NSC siRNA. At 3-day post-transfection, the cell culture supernatants were used to infect nave Huh7.5 cells. At 3-days p.i., the level of HCV NS3 protein and positive-strand RNA were quantified by Western blot analysis and RPA, respectively, as shown in FIG. 8A. FIGS. 8A and 8B show that the levels of HCV NS3 and positive-strand RNA were proportionally lowered by increasing concentrations of apoE-specific siRNA but not NSC siRNA, demonstrating that the knockdown expression of apoE by specific siRNA efficiently suppressed HCV production. The infectious HCV titers in the culture supernatant were also reduced by apoE-specific siRNA by 100 times at 50 nM concentration as shown in FIG. 8C. To further determine whether the apoE-specific siRNA suppressed HCV assembly and production, HCV vRNA extracted from cell culture supernatants, which reflects the total HCV RNA-containing particles, was quantified by RPA. FIGS. 9A-9B show that apoE-specific siRNA but not NSC siRNA remarkably reduced HCV vRNA levels (undetectable at 50 nM by RPA) in the culture supernatants, demonstrating that the knockdown of apoE by specific siRNA efficiently blocked HCV production. Additionally, the intracellular HCV virions were prepared by freezing and thawing cells that were infected with HCV and transfected with siRNA. The infectivity of intracellular HCV virions was measured by the level of HCV positive-strand RNA by RPA in the infected Huh7.5 cells. The infectious titers of intracellular HCV virions were determined by immunofluorescence assay (IFA). FIGS. 10A-10C show that apoE-specific siRNA reduced the infectivity of intracellular HCV virions by more than 70% as illustrated in FIGS. 10A and 10B and infectious titer by more than 10-fold as illustrated in FIG. 10C at concentrations up to 50 nM. To rule out the possibility of whether apoE-specific siRNA affects HCV RNA replication, Huh7 cells carrying a subgenomic HCV RNA, which can replicate in the cell but does not produce HCV virions, were transfected with apoE-specific or NSC siRNA. At 3-day post-transfection, HCV RNAs were extracted with Trizol reagent, and the levels of HCV positive-strand RNA were quantified by RPA. As shown in FIG. 11, apoE-specific siRNA did not significantly affect HCV RNA replication, demonstrating that apoE is important for HCV virion assembly but not for RNA replication. Taken together, these findings demonstrate that apoE-specific siRNA efficiently knocked down apoE expression and therein suppressed HCV virion assembly and production.

With reference to FIGS. 12A and 12B, the effects of microsomal triglyceride transfer protein (MTP)- and Sterol O-acyltransferase (SOAT, also named Acyl CoenzymeA:cholesterol Acyltransferase-ACAT)-specific siRNAs on HCV production were studied. HCV-producing cells, were transfected with increasing concentrations (nM) of siRNAs against MTP and ACAT, respectively. At 5 days post-transfection, cell culture supernatant was used to infect naïve Huh7.5 cells. At 3 days p.i., total cellular RNAs were extracted and the levels of positive-stranded HCV RNA were determined by RPA. Results show that MTP- and ACAT-specific siRNAs reduced HCV production by up to 60% at concentrations up to 50 nM as illustrated in FIG. 12B.

These results are consistent with above-described findings that apolipoproteins incorporated into HCV virions are required for HCV infectivity and virion assembly. Inhibition of apolipoprotein production/assembly and secretion by apoE-, apoC-I-, ACAT, and MTP-specific siRNAs reduced HCV virion production and infection. The studies described in the above example indicate that siRNAs against apolipoproteins and lipoprotein biosynthetic pathways can be used as antiviral agents against HCV infection.

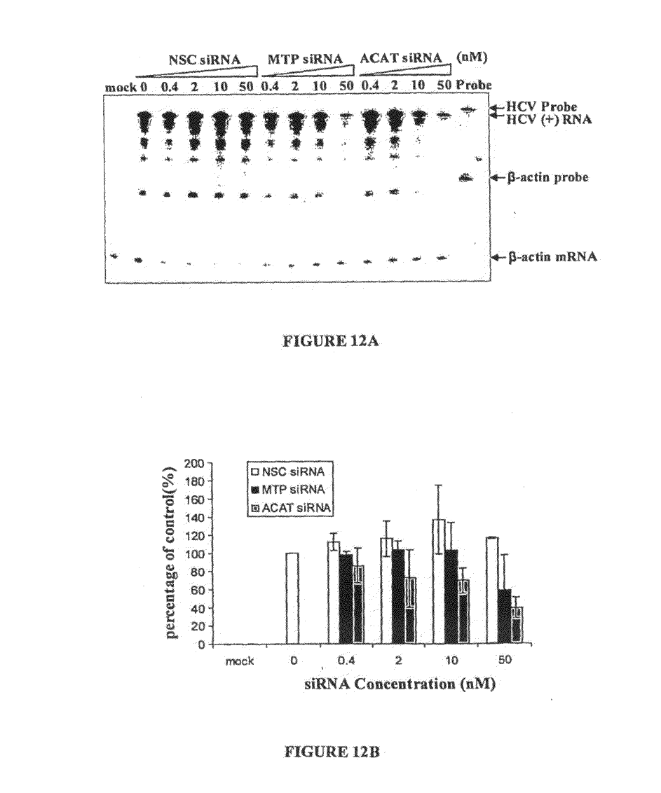

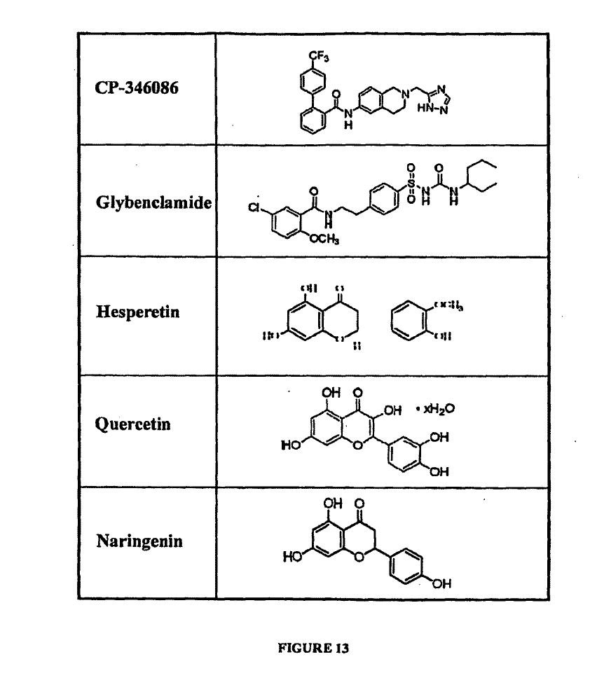

Inhibition of HCV assembly, production, and infectivity by small molecular compounds against lipoprotein production and secretion: In order to determine whether small molecular inhibitors of lipoprotein assembly and secretion can suppress HCV production and infection, several small molecule inhibitors, CP-346086, glybenclamide, hesperetin, and quercetin, of the intracellular lipid and lipoprotein synthesis, assembly, and secretion were examined. The chemical structures of these inhibitors are set forth in FIG. 13. These inhibitors are examples to provide a proof-of-concept that lipid and lipoprotein synthesis, assembly, and secretion are unique targets for antiviral drugs against HCV production and infection. Other drugs include ACAT, MTP, Cholesteryl Ester Transfer Protein (CETP), sterol response element-binding proteins (SREBPs) and SREBP-cleavage activating proteins (SCAP) inhibitors that are currently in preclinical development and/or clinical trials for treating cardiovascular diseases and lowering cholesterol.

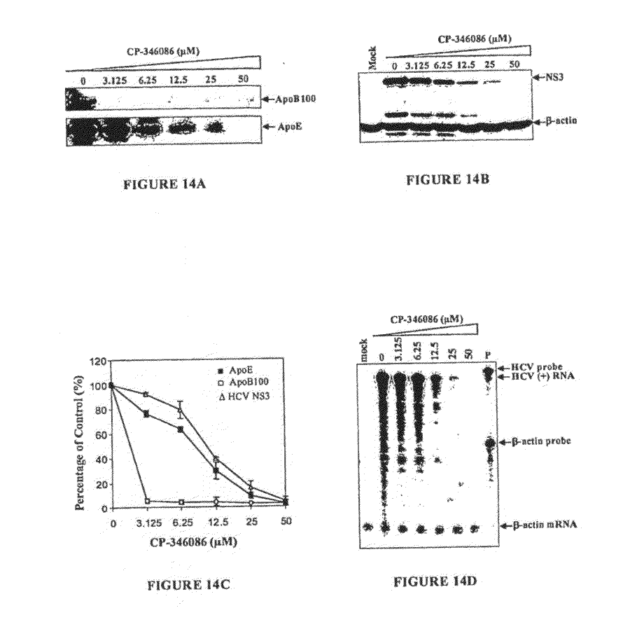

With reference to FIGS. 14A-14F, studies were carried out to determine the effects of an MTP inhibitor, CP-346086, on lipoprotein production/secretion and HCV production. CP-346086 is a potent MTP inhibitor that efficiently blocks apoB and triglyceride secretion with an IC₅₀ of 2.6 nM in HepG2 cells. To determine the effect of CP-346086 on HCV production, Huh7.5 cells were infected with HCV at 37° C. for 2 hr and then incubated with varying concentrations of CP-346086 for 4 days. The levels of apoB and apoE in the media were determined by Western blotting. As shown in FIG. 14A, apoB- and apoE-containing lipoproteins displayed different sensitivity to CP-346086 treatment. CP-346086 almost completely blocked apoB secretion at 1 μM, but did not reduce apoE secretion until above 3 μM. The levels of HCV virions released into the media were initially determined by infectivity assay in cell culture. The naïve Huh7.5 cells were infected with HCV-containing media. At 3 days p.i., the levels of HCV NS3 protein and positive-stranded RNA were quantified by Western blotting and RPA, respectively. Consistent with the levels of apoE reduction by CP-346086, the levels of both HCV NS3 protein and positive-stranded RNA were proportionally reduced by increasing concentrations of CP-346086, resulting in >95% reduction at 50 μM as illustrated in FIGS. 14B, 14C, and 14D. Similarly, CP-346086 lowered the infectious HCV titers by 1, 2, 3, and 4 orders of magnitude (10, 100, 1,000, and 10,000 times) at concentrations of 6.25, 12.5, 25, and 50 μM, respectively, as illustrated in FIG. 14E. To exclude a possible inhibition of HCV RNA replication, CP-346086 was used to treat Huh7.5 cells harboring a subgenomic HCV replicon of genotype 2a, which replicated in the cell but did not produce HCV virions. As shown in FIG. 14F, CP-346086 had no significant effect on HCV RNA replication. These findings demonstrate that inhibitors blocking apoE production and secretion can be used as antiviral agents against HCV infection.

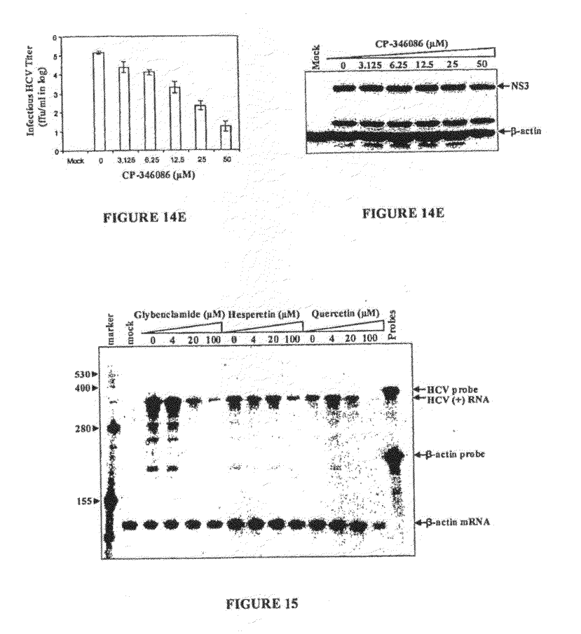

With reference to FIG. 15, three commercial inhibitors, glybenclamide, hesperetin, and quercetin, of lipoprotein assembly and secretion all significantly decreased the levels of HCV virion production. These inhibitors were used to treat HCV-infected cells. The culture supernatants were then used to infect naïve Huh7.5 cells. At 3 days p.i., the levels of positive-strand HCV RNA were determined by RPA. Results show that all these inhibitors reduced HCV production to certain degrees. These findings demonstrate that inhibitors of lipoprotein assembly and secretion suppressed HCV production, which can be potentially used as antiviral drugs against HCV replication and infection. Our findings also suggest that other inhibitors of ACAT, MTP, CETP, SREBP, and SCAP can be potential anti-HCV drugs.

Inhibition of HCV infection by human HDL, LDL, and VLDL: As described above, HCV virions contain human apolipoproteins. Human apolipoproteins were shown to bind the low-density lipoprotein (LDL) receptor and SR-BI, which serve as receptors/co-receptors for HCV infection. A study was conducted to determine whether recombinant apolipoprotein E and human lipoproteins of various densities would inhibit HCV infection.

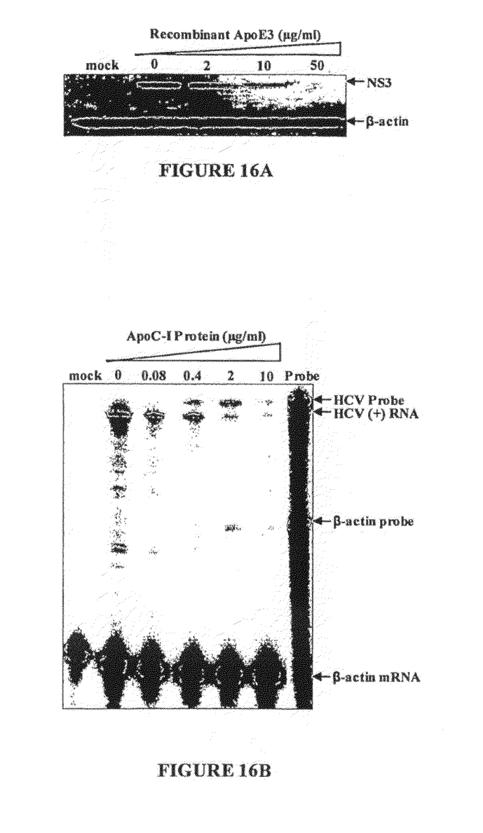

With reference to FIGS. 16A and 16B, the effect of recombinant apolipoprotein E3 (apoE3) and purified apoC-I on HCV infection was investigated. Huh7.5 cells were infected with HCV in the presence of increasing concentrations (μg/ml) of apoE3 or apoC-I, as indicated on the top of the figures. At 3-hr p.i., the HCV-infected cells were washed with 1×PBS twice and were incubated with medium for 3 days. With reference to FIG. 16A, the levels of HCV NS3 protein in the cells infected with HCV in the presence of apoE3 were then determined by Western blot analysis using an NS3-specific monoclonal antibody and a cellular beta-actin protein as an internal control. With reference to FIG. 16B, HCV positive-strand RNA in the cells infected with HCV in the presence of apoC-I were quantified by RPA. Results demonstrate that ApoE3 and ApoC-I inhibited HCV infection in a dose-dependent manner.

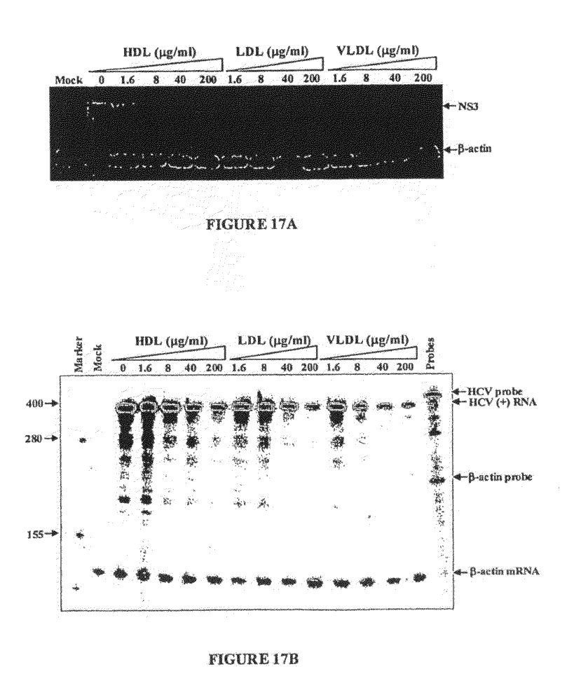

With reference to FIGS. 17A and 17B, the effect of human HDL, LDL, and VLDL on inhibition of HCV infection was studied. Huh7.5 cells were infected with HCV in the presence of increasing concentrations (μg/ml) of lipoproteins, as indicated on the top of the figures. At 3 hr p.i., HCV/lipoproteins were removed, and cells were washed twice with 1×PBS and incubated with DMEM. With reference to FIG. 17A, at 3 days p.i., cells in one set were lysed for Western blot analysis, and with reference to FIG. 17B, the cells in the other set were lysed for RNA extraction with Trizol reagent and RNA quantification by RPA. The levels of positive-stranded HCV RNA were determined by RPA.

The human HDL, LDL, and VLDL were all found to inhibit HCV infection in a dose-dependent manner when incubated with HCV RNA-containing particles during infection. HDL, LDL, and VLDL are known to contain increasing amounts of apoE. These findings suggest that human lipoproteins containing apoE compete the binding of the HCV receptors/co-receptors with HCV virions.

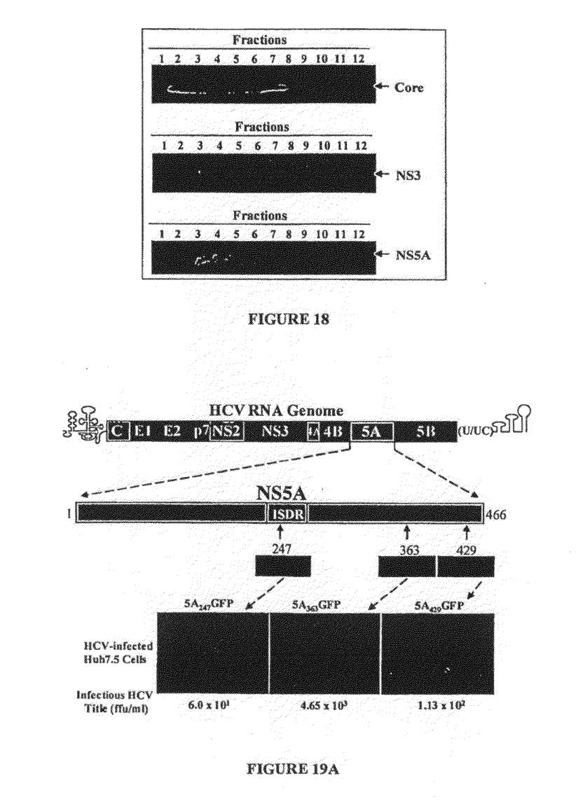

To determine whether HCV RNA-containing particles produced by cell culture in vitro contain viral nonstructural proteins, Western blot analysis was performed using HCV core-, NS3, and BS5A-specific monoclonal antibodies. With reference to FIG. 18, HCV NS3 and NS5A proteins besides HCV capsid protein Core (C) were detected in HCV RNA-containing particles in fractions 3-7, consistent with their infectivity, as shown in FIG. 3B and FIG. 3C. These results demonstrate that HCV nonstructural proteins are incorporated into HCV virions, which suggest that HCV nonstructural proteins play important roles in HCV virion assembly and production.

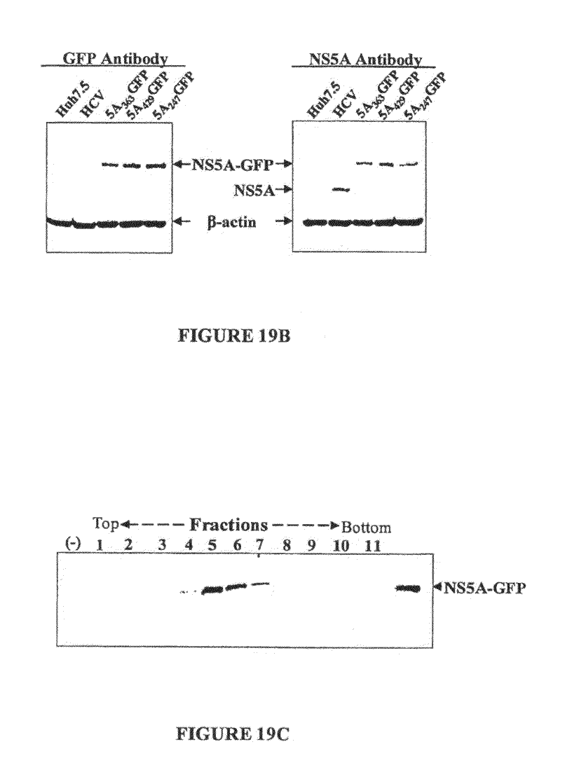

With reference to FIG. 19A, infectious HCVs expressing a fusion protein between NS5A and green fluorescence protein (GFP) were genetically constructed. The coding sequence of GFP was inserted into the NS5A gene, where the insertion site was determined by a random insertion mutagenesis method using a transposon-based EZ-Tn5 In-Frame Linker Insertion Kit from EPICENTRE Biotechnologies. Three sites at amino acid 247, 363, and 429 in NS5A were found to tolerate insertions without ablation of HCV production. The GFP-coding sequence was then inserted into these sites of NS5A gene in the infectious HCV RNA genome. The resulted infectious HCVs were viable and express an NS5A-GFP fusion protein, as determined by the presence of GFP in the HCV-infected Huh7.5 cells, appearing as bright spots in FIG. 19A. The infectious HCV titers, as determined by IFA as foci-forming units per milliliter of cell culture supernatant (ffu/ml) and as indicated at the bottom of FIG. 19A, were lower than wild type HCV by up to 100 times. Wild type HCV are generally around 10⁵ ffu/ml. These findings suggest that NS5A is important for HCV assembly and virion production. With reference to FIG. 19B, the NS5A-GFP fusion protein was detected by Western blot using a GFP-specific monoclonal antibody in the HCV-infected cells in comparison to the presence of NS5A in the wild type HCV-infected cells. With reference to FIG. 19C, the NS5A-GFP fusion protein was shown to be efficiently incorporated into infectious HCV particles. Collectively, these findings demonstrate for the first time that HCV nonstructural proteins such as NS3, NS4B, NS5A, and likely NS2 are assembled in the infectious HCV virions, providing targets and means to screen for inhibitors that block the incorporation of HCV nonstructural proteins and their fusion forms with reporter proteins (e.g., NS5A, NS5A-luciferase, NS5A-GFP, NS5A-alkaline phosphotase, etc.) into HCV virions.

A screening method for identifying a candidate agent as an actual anti-Hepatitis C virus agent comprises treating the cells with the candidate anti-Hepatitis C virus agent, and determining whether the levels Hepatitis C virus nonstructural proteins and their fusion proteins with reporters (reporter activity if it is an enzyme) in a cell culture supernatant is below a predetermined level and/or control. The Hepatitis C virus nonstructural proteins and/or their fusion forms with reporter proteins in the cell culture supernatant is measured by determining the Hepatitis C virus nonstructural protein levels and/or reporter activity when fused with reporter proteins as fusion proteins in the cell culture supernatant.

It will be apparent to those skilled in the art that various modifications and variations can be made in the present invention without departing from the scope or spirit of the invention. Other embodiments of the invention will also be apparent to those skilled in the art from consideration of the specification and practice of the invention disclosed herein. It is intended that the specification and example be considered as exemplary only, and not intended to limit the scope and spirit of the invention.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in the Specification and Claims are approximations that can vary depending upon the desired properties sought to be determined by the present invention. Notwithstanding that the numerical ranges and parameters setting forth the broad scope of the invention are approximations, the numerical values set forth in the Examples are reported as precisely as possible. Any numerical value, however, inherently contain certain errors necessarily resulting from the standard deviation found in their respective testing measurements.

We claim:

1. A composition for treating and/or preventing Hepatitis C virus infection in a patient, comprising a purified antibody which recognizes apolipoprotein E and/or apolipoprotein C-I, wherein said antibody is one of monoclonal, polyclonal, and humanized recombinant.

2. A method of treating and/or preventing Hepatitis C virus infection in a patient, comprising: providing the composition of claim 1; and administering to the patient an effective amount of the composition.

3. A composition for treating and/or preventing Hepatitis C virus infection in a patient, comprising: an siRNA specific for a apolipoprotein.

4. The composition of claim 3, wherein a target apolipoprotein is selected from a group, including but not restricted to apolipoprotein E.

5. A method of treating and/or preventing Hepatitis C virus infection in a patient, comprising: providing the composition of claim 3; and administering to the patient an effective amount of the composition.

6. A composition for treating and/or preventing Hepatitis C virus infection in a patient, comprising an siRNA specific for a lipoprotein biosynthetic gene, selected from a group, including acyl coenzyme A:cholesterol acyltransferase, Cholesteryl Ester Transfer Protein, and microsomal triglyceride transfer protein.

7. A method of treating and/or preventing Hepatitis C virus infection in a patient, comprising: providing the composition of claim 6; and administering to the patient an effective amount of the composition.

8. A method of treating and/or preventing Hepatitis C virus infection in a patient, comprising: providing a composition including a small molecular inhibitor of lipoprotein biosynthesis; and administering to the patient an effective amount of the composition.

9. The method of claim 8, wherein the small molecular inhibitor of lipoprotein biosynthesis is an inhibitor of a lipoprotein biosynthetic gene, selected from a group, comprising acyl coenzyme A:cholesterol acyltransferase, Cholesteryl Ester Transfer Protein, microsomal triglyceride transfer protein, and the gene responsible for apoE lipoprotein synthesis, assembly and/or secretion.

10. The method of claim 8, wherein the small molecular inhibitor of lipoprotein biosynthesis is selected from a group, including CP346086, torcetrapib, glybenclamide, hesperetin, quercetin, and naringenin.

11. A method of treating and/or preventing Hepatitis C virus infection in a patient, comprising: providing a composition capable of suppressing apolipoprotein E levels in the patient; and administering to the patient an effective amount of the composition.

12. The method of claim 11, wherein the composition includes an agent selected from the group including: an agent that is capable of pre-transcriptionally suppressing apolipoprotein E levels; an agent that is capable of post-transcriptionally suppressing apolipoprotein E levels; an agent that is capable of post-translationally suppressing apolipoprotein E levels; and an agent that is capable of post-translationally affecting the modification of apolipoprotein E.

13. A screening method for identifying anti-Hepatitis C virus agents, comprising: providing cells in a culture; treating said cells with a candidate anti-Hepatitis C virus agent; measuring apolipoprotein E expression in the cells and/or supernatant of said cell culture; and identifying the candidate agent as an actual anti-Hepatitis C virus agent when apolipoprotein E gene expression in the cells and/or said supernatant is below a predetermined level.

14. The method of claim 13, wherein the apolipoprotein E gene expression in the cells and/or in the cell culture supernatant is measured by determining the apolipoprotein E mRNA and/or protein.

15. A screening method for identifying anti-Hepatitis C virus agents, comprising: providing cells in culture; treating the cells with a candidate anti-Hepatitis C virus agent; measuring the levels of one or more of Hepatitis C virus nonstructural proteins or reporter activity that is fused with the nonstructural proteins in the cell culture supernatant; and identifying the candidate agent as an actual anti-Hepatitis C virus agent when Hepatitis C virus nonstructural proteins and/or reporter activity, in the case of a fusion protein, if the cell culture supernatant is below a predetermined level and/or control.
